(19) United States
(12) Patent Application Publication
(10) Pub. No.: US 2015/0038911 A1
(43) Pub. Date: Feb. 5, 2015

(54) MICRONEEDLE ADAPTER FOR DOSED DRUG DELIVERY DEVICES

(71) Applicant: NANOPASS TECHNOLOGIES LTD, Nes Ziona (IL)

(72) Inventors: Yotam Levin, Nes Ziona (IL); Yehoshua Yeshurun, Haifa (IL); Nadav Agian, Kfar Yona (IL); Barak Panga, Moshav Ptahia (IL); Yoel sefi, Kibbutz Malkia (IL); Gal Admati, Kibbutz Dorot (IL)

(73) Assignee: NANOPASS TECHNOLOGIES LTD, Nes Ziona (IL)

(21) Appl. No.: 14/373,634

(22) PCT Filed: Jan. 24, 2013
(86) PCT No.: PCT/IB2013/050617

§ 371 Date: Jul. 22, 2014

Related U.S. Application Data
(60) Provisional application No. 61/589,882, filed on Jan. 24, 2012.
(60) Provisional application No. 61/598,364, filed on Feb. 14, 2012.

(51) Int. Cl. A61M 5/24; A61M 37/00

(52) U.S. Cl. CPC A61M 5/2466; A61M 37/0015; A61M 2005/2474; A61M 2210/04; A61M 2037/0023
USPC 604/173; 604/205

(57) ABSTRACT

An adapter (50) for achieving intradennal dosed delivery of a liquid by use of a dosed drug delivery device including a reservoir having a pierceable septum, the adapter having a connector including an attachment configuration (22) for sliding a recess of the adapter onto a projection of the dosed drug delivery device and a hollow needle (25) deployed for piercing the septum. At least one microneedle (10) projects from the connector. A flow path arrangement (28) interconnects the needle and the hollow microneedle(s). The recess is shaped to restrict the microneedle(s) to only a finite number of rotational orientations relative to the axis of the device. The connector and/or the needle is adapted for application of a sealant to prevent leakage of the liquid.

### FIELD AND BACKGROUND OF THE INVENTION

The present invention relates to drug deliver devices and, in particular, it concerns a microneedle adapter for use with dosed drug delivery devices.

Dosed drug delivery devices, often referred to as “pen injectors,” are commonly used by diabetics for routine injection of insulin. Similar devices are also used for the delivery of hormones. Dosed drug delivery devices are a preferred means of delivery wherever the volume of drug delivered needs to be variable but accurate, small and frequently administered. Use of the term “pen injector” probably stems from the elongated pen-like form of many of the commercially available devices. However, unless otherwise specified, the term “pen injector” will be used herein interchangeably with the term “dosed drug delivery device” to refer generically to any and all free-standing portable device containing a plurality of doses of a therapeutic liquid which can be operated by a patient for self-injection to deliver metered doses of the liquid to the patient's body on a plurality of occasions. There are various kinds of pen injectors which may be variously classified according to different structural or functional features, such as: devices employing replaceable cartridges and devices which are disposed of when the contents are finished; devices with fixed dosage units or with various dialing and dosing features; devices with different flow activation mechanisms, ergonomics and design, reservoir systems and volume requirements etc.

Pen injectors are used with dedicated replaceable needle assemblies, referred to herein for convenience as “pen needles”. Commercially available pen needles known to the inventors all target the subcutaneous (SC) fatty layer and make use of tubular metal components (hypodermic stainless steel needles). Commercially available pen needles typically have lengths ranging from 1 mm to 25 mm.

Pen needles are configured to satisfy several requirements unique to pen injectors. On one side, they feature a connector for reversibly connecting to a liquid reservoir within the pen injector. The connector typically includes a hollow needle deployed for piercing a septum (resilient self-sealing membrane) integrated with the liquid cartridge, and an attachment configuration such as a threaded collar for attachment to the pen injector. On the other side, the pen needle features the skin-penetrating needle. The septum-piercing needle and the skin-penetrating needle are typically implemented as opposite ends of a single double-ended needle. The two ends typically have different point shapes, with the rear end configured to avoid coring of a hole in the septum and the front end shaped to minimize pain on penetration through the skin. This renders the double ended needle complex to manufacture. On the other hand, since a single continuous needle is used, there is typically no requirement of sealing between the needle and the surrounding connector body, often allowing the structure to be assembled without the sealing glue required for other hypodermic applications, and the “dead volume” of the needle is very small. For all of the above reasons, design considerations for pen needles are significantly different from those of other hypodermic needles, and such needles have attained a distinct status in the art, often being produced by Specialist companies which deal exclusively with pen needles and other pen injector related accessories.

Miniature needles used for pen injectors typically project a minimum of 1 millimeter. In the case of a miniature needle of conventional hypodermic type (i.e., a metal tube formed with a beveled end), the bevel of the needle tip itself typically has a length of at least 0.8 mm, making it impossible to achieve sealed fluid delivery to penetration depths less than 1 mm.

In some published documents, it has been proposed to use “microneedles” as a delivery interface for pen injectors. For the purpose of the present description and claims, the term “microneedle” in its broadest sense is used to refer to a projecting structure with a projecting length of less than 1 millimeter. Examples of such documents include US patent application publication nos. US 2003/0050602 to Pettis and US 2003/0181863 to Ackley et al. Theoretically, application of microneedles to pen injectors promises various advantages attributed to intradermal delivery including, but not limited to, altered kinetics (depending on the formulation and the exact injection site, either accelerated absorption, such as may be beneficial for insulin delivery or delayed absorption, for example if a slow release formulation is used), improved response (for example intradermal delivery of vaccines may enhance immune response, allow for smaller doses, potentially lesser booster shots, better vaccination, etc), reduced trauma (since microneedles are smaller than conventional hypodermic needles), and minimally painful or painless injections. The last feature, in particular, is considered highly significant, possibly increasing patient compliance, improving quality of life, improving disease control and reducing expenses on treatment of disease complications. This is particularly relevant in the case of insulin injections for treatment of diabetes due to the direct relation between long term control of blood glucose levels and the prevalence of long term complications.

In practice, implementations of microneedles for pen injectors are not straightforward due to a number of practical problems. A first major problem of many microneedle designs relates to mechanical weakness of the microneedles which tend to fracture on contact with the skin, particularly when exposed to shear forces due to lateral movement A further problem is that the highly elastic skin barrier tends to deform around the microneedles without the microneedles penetrating through the stratum corneum (SC). An additional problem is that of leakage around the microneedles' point of insertion and/or ejection of the needles by back-pressure generated during injection. Many designs are also prone to blockage of the bores of hollow microneedles due to punching-out of a plug of tissue during insertion through the skin.

Solutions to the aforementioned problems have been suggested in the context of applications such as infusion sets and syringes. Particularly, reference is made to a particularly advantageous robust microneedle structure as taught by U.S. Pat. No. 6,533,949, and to various microneedle insertion techniques as taught by PCT Patent Application Publication Nos. WO 03/074102 A2 and WO 2005/049107 A2, and in US Patent Application Publication No. US 2005/0209566 A1, these four publications mentioned in this paragraph all being hereby incorporated by reference in their entirety. However, these solutions have not previously been adapted to address the particular requirements of pen injectors. Furthermore, given the unique design considerations for pen needles, and the distinct status of pen needles as established in the art, such adaptations are not readily apparent to a person having ordinary skill in the art.

There is therefore a need for an adapter for achieving intradermal dosed delivery of a liquid by use of a dosed drug delivery device.

### SUMMARY OF THE INVENTION

The present invention is an adapter for achieving intradermal dosed delivery of a liquid by use of a dosed drug delivery device.

According to the teachings of the present invention there is provided a first adapter for achieving intradermal dosed delivery of a liquid by use of a dosed drug delivery device, the dosed drug delivery device including a reservoir having a pierceable septum, the adapter comprising: (a) a connector including an attachment configuration for attachment to the dosed drug delivery device and a hollow needle deployed for piercing the septum; (b) a liquid delivery interface mechanically linked to the connector, the liquid delivery interface including a substantially straight skin contact edge and a linear array of hollow microneedles deployed substantially adjacent to, and arrayed substantially parallel to, the skin contact edge, the microneedles projecting away from the skin contact edge; and (c) a flow path arrangement interconnecting the needle and the array of hollow microneedles.

According to a further feature of the present invention, each of the microneedles has a height, and wherein a distance between the skin contact edge and each of the microneedles is no greater than the height of the microneedles.

According to a further feature of the present invention, the substantially straight skin contact edge is formed as an edge of a block of material, the block of material being integrally formed with at least part of the attachment configuration.

According to a further feature of the present invention, an extensional direction of the hollow needle of the connector defines a primary flow axis, and wherein each of the hollow microneedles includes a flow channel defining an injection direction, the injection direction being inclined relative to the primary flow axis by an angle of at least 20 degrees.

According to a further feature of the present invention, the injection direction is inclined relative to the primary flow axis by an angle of between 30 degrees and 150 degrees.

According to a further feature of the present invention, the injection direction is inclined relative to the primary flow axis by an angle of about 90 degrees.

According to a further feature of the present invention, the hollow microneedles are integrally formed with a substrate.

According to a further feature of the present invention, the substrate has a substantially planar surface, and wherein each of the microneedles is fainted by at least one wall standing substantially upright from the substantially planar surface and an inclined surface intersecting with the at least one wall.

According to a further feature of the present invention, each of the microneedles has a flow channel passing through the substrate and intersecting with the inclined surface.

According to a further feature of the present invention, the microneedles are formed from silicon.

There is also provided according to the teachings of the present invention, a combination of the aforementioned adapter with a dosed drug delivery device, the combination further including a dosed drug delivery device having a liquid reservoir including a pierceable septum, the adapter being connected to the dosed drug delivery device so that the hollow needle pierces the septum thereby bringing the microneedles into flow connection with contents of the reservoir.

According to a further feature of the present invention, the reservoir contains a quantity of insulin. Alternatively, the reservoir contains a quantity of a fertility hormone. In a further alternative, the reservoir contains a quantity of a growth hormone. In yet a further alternative, the reservoir contains a quantity of a vaccine.

There is also provided according to the teachings of the present invention, a second adapter for achieving intradermal dosed delivery of a liquid by use of a dosed drug delivery device, the dosed drug delivery device including a reservoir having a pierceable septum, the adapter comprising: (a) a connector including an attachment configuration for attachment to the dosed drug delivery device and a hollow needle deployed for piercing the septum; (b) liquid delivery interface mechanically linked to the connector, the liquid delivery interface including a substantially straight skin contact edge and at least one hollow microneedle deployed substantially adjacent to the skin contact edge, the at least one microneedle projecting away from the skin contact edge; and (c) a flow path arrangement interconnecting the needle and the at least one hollow microneedle.

Furthermore, according to the present invention there is provided a third adapter for achieving intradermal dosed delivery of a liquid by use of a dosed drug delivery device, the dosed drug delivery device including a reservoir having a pierceable septum, the adapter including: (a) a connector including: (i) an attachment configuration for attachment to the dosed drug delivery device by sliding the adapter onto the dosed drug delivery device so that a recess of the adapter mates with a matching projection of the dosed drug delivery device, and (ii) a hollow needle deployed for piercing the septum; (b) at least one hollow microneedle projecting from the connector; and (c) a flow path arrangement interconnecting the needle and the at least one hollow microneedle; wherein the recess is shaped to restrict the at least one microneedle to one of a finite number of rotational orientations relative to an axis of the dosed drug delivery device when the adapter is attached to the dosed drug delivery device.

Furthermore, according to the present invention there is provided a fourth adapter for achieving intradermal dosed delivery of a liquid by use of a dosed drug delivery device, the dosed drug delivery device including a reservoir having a pierceable septum, the adapter including: (a) a connector including an attachment configuration for attachment to the dosed drug delivery device and a hollow needle deployed for piercing the septum; (b) at least one hollow microneedle projecting from the connector; and (c) a flow path arrangement interconnecting the needle and the at least one hollow microneedle; wherein at least one of the connector and the needle is adapted for application of a sealant to cover the flow path arrangement and at least a portion of the needle so as to prevent leakage of the liquid from the flow path arrangement.

Furthermore, according to the present invention there is provided a fifth adapter for achieving intradermal dosed delivery of a liquid by use of a dosed drug delivery device, the dosed drug delivery device including a reservoir having a pierceable septum, the adapter including: (a) a connector including an attachment configuration for attachment to the dosed drug delivery device and a hollow needle deployed for piercing the septum; (b) a linear array of hollow microneedles projecting from the connector; and (c) a flow path arrangement, interconnecting the needle and the at least one hollow microneedle, that is wider, in a direction transverse to a direction of a flow of the liquid from the needle to the array of microneedles, than both the needle and the array of microneedles.

Furthermore, according to the present invention there is provided a sixth adapter, for achieving intradermal dosed delivery of a liquid, including: (a) a connector including an attachment configuration for attachment to a dosed drug delivery device; (b) a linear array of hollow microneedles projecting from the connector; wherein at least a portion of the connector adjacent the linear array of microneedles is wider in a direction parallel to the linear array of microneedles than in a direction perpendicular to the linear array of microneedles, the adapter further including: (c) a cap, shaped to conform to the connector and to slide onto the connector to cover the microneedles and at least a portion of the portion of the connector that is adjacent to the microneedles, an edge of the cap that receives the connector, when the cap is slided onto the connector, being recessed on a portion thereof that is parallel to the linear array of microneedles relative to a remainder thereof.

Furthermore, according to the present invention there is provided a cap, for a dosed drug delivery device from which at least one microneedle projects, the cap including: (a) a cap body, shaped to conform to at least a portion of the dosed drug delivery device when the cap is placed onto the dosed drug delivery device to cover the at least one microneedle; (b) a cap cover, at a distal end of the cap body, that substantially seals the distal end of the cap body; and (c) a mechanism, in the cap body, for reversibly urging the dosed drug delivery device away from the cap cover; wherein the cap cover includes an aperture from which the at least one microneedle protrudes when the dosed drug delivery device is urged towards the cap cover.

Furthermore, according to the present invention there is provided a fluid delivery device including: (a) a hollow needle; (b) a fitting for operationally connecting the needle to a syringe; (c) a cap that conceals the needle and that includes an aperture; and (d) a mechanism that reversibly urges the aperture away from the needle, thereby preventing the needle from emerging from the aperture unless an opposing force is applied to the fluid delivery device via the fitting.

The third adapter of the present invention is similar to the second adapter, with the attachment configuration being attached to the dosed drug delivery device by sliding the adapter onto the dosed drug delivery, device so that a recess of the adapter mates with a matching projection of the dosed drug delivery device. The recess is shaped to restrict the microneedle(s) to one of a finite number of rotational orientations, and preferably to a single such rotational orientation, relative to the axis of the dosed drug delivery device when the adapter is attached to the dosed drug delivery device.

Preferably, the liquid delivery interface includes a linear array of the microneedles.

A combination of the third adapter with a dosed drug delivery device also is provided, the combination further including a dosed drug delivery device having a liquid reservoir including a pierceable septum, the adapter having been connected to the dosed drug delivery device by sliding the adapter onto the dosed drug delivery device so that the recess mates with the projection and so that the hollow needle pierces the septum thereby bringing the microneedle(s) into flow connection with contents of the reservoir.

Preferably, the attachment configuration includes a mechanical arrangement for inhibiting removal of the ad pier from the dosed drug delivery device after the adapter has been attached to the dosed drug delivery device.

The fourth adapter of the present invention also is similar to the second adapter, with either or both of the connector or the hollow needle is/are adapted for application of a sealant to cover the flow path arrangement and at least part of the needle to prevent leakage of the liquid from the flow path arrangement. For example, the connector could include a port for introducing the sealant into the interior of the connector, or the outer surface of the needle could be formed in a manner that promotes bonding thereto of the sealant.

The fifth adapter of the present invention also is similar to the second adapter, with the flow path arrangement being wider in a direction transverse to the direction of the flow of the liquid from the needle to the array of microneedles, than both the needle and the array of microneedle(s).

The sixth adapter of the present invention includes the connector and. the liquid delivery interface of the second adapter. At least the portion of the connector that is adjacent to the array of microneedles is wider in the direction parallel to the array of microneedles than. in the direction perpendicular to the array of microneedles. The adapter also is provided with a cap that is shaped to conform to the connector and to slide onto the connector to cover the microneedles and the adjacent portion of the connector. The edge of the cap that receives the connector when the cap is slided onto the connector is recessed, relative to the rest of the edge, on the portion of the edge that is parallel to the array of microneedles.

The scope of the present invention also includes a cap for a dosed drug delivery device equipped with an adapter, such as the adapters of the present invention, from which one or more microneedles project. The cap includes a cap body and a cap cover. The cap body is shaped to conform to at least a portion of the dosed drug delivery device when the cap is placed on the dosed drug delivery device to cover the microneedle(s). The cap cover, at the distal end of the cap, substantially seals the cap body. The cap also includes a mechanism, in the cap body for reversibly urging the dosed drug delivery device away from the cap cover. The cap cover includes an aperture from which the microneedle(s) protrude(s) when the dosed drug delivery device is urged towards the cap cover.

Preferably, at least part of the outer surface of the cap cover is roughened to apply tension to skin into which liquid is being delivered via the microneedle(s) as the microneedle(s) protrude(s) from the aperture. Most preferably, the cap cover is substantially planar and is tilted so that the roughened portion of the cap cover contacts the skin before the microneedle(s) when the dosed drug delivery device, with the cap covering the liquid delivery interface, is applied to the skin to deliver the liquid into the skin via the microneedle(s) as the microneedle(s) protrude(s) from the aperture.

The scope of the present invention also includes a fluid delivery device that includes a hollow needle such as a microneedle, a fitting for operationally connecting the needle to a syringe, a cap that conceals the needle and that includes an aperture, and a mechanism that reversibly urges the aperture away from the needle, so that the needle is prevented from emerging from the aperture unless an opposing force is applied to the fluid delivery device via the fitting.

### BRIEF DESCRIPTION OF THE DRAWINGS

The invention is herein described, by way of example only, with. reference to the accompanying drawings, wherein:

FIG. 1 is an isometric view of a preferred form of a linear array of microneedles for use in the adapters of the present invention;

FIG. 2A is a exploded isometric view of a first embodiment of an adapter, constructed and operative according to the teachings of the present invention, for use with a dosed drug delivery device to achieve intradermal dosed delivery of a liquid;

FIG. 2B is a isometric partially-cut-away view of the adapter of FIG. 2A as assembled prior to use;

FIG. 2C is a cross-sectional view taken through the adapter of FIG. 213 after removal of protective covers;

FIG. 3 is a cross-sectional view similar to FIG. 2C taken through a second embodiment of an adapter, constructed and operative according to the teachings of the present invention, for use with a dosed drug delivery device to achieve intradermal dosed delivery of a liquid;

FIG. 4A is a side view of the adapter of FIG. 2B assembled on a pen injector ready for use;

FIG. 4B is an enlarged view of the region of FIG. 4A including the adapter;

FIG. 5A is a side view of the adapter of FIG. 3 assembled on a pen injector ready for use;

FIG. 5B is an enlarged view of the region of FIG. 5A including the adapter;

FIG. 6A is a view similar to FIG. 4B after interfacing of the adapter with the skin of a user;

FIG. 6B is a view similar to FIG. 6A after injection of a dose of the liquid;

FIG. 7A is a view similar to FIG. 5B after interfacing of the adapter with the skin of a user; and

FIG. 7B is a view similar to FIG. 7A after injection of a dose of the liquid;

FIGS. 8A and 8B show an adapter with an elliptical recess and a corresponding pen injector with an elliptical projection;

FIGS. 9A and 9B show an adapter with a rectangular recess and a corresponding pen injector with a rectangular projection;

FIGS. 10A and 10B show an adapter with a hexagonal recess and a corresponding pen injector with a hexagonal projection;

FIGS. 11A and 11B show an adapter with a notched recess and a corresponding pen injector with a tabbed projection;

FIGS. 12A and 12B show an adapter with a truncated cylindrical recess and a corresponding pen injector with a truncated cylindrical projection;

FIGS. 13A and 13B show an adapter with an isosceles triangular recess and a corresponding pen injector with an isosceles triangular projection;

FIGS. 14A and 14B show an adapter and a pen injector configured to keep the adaptor from sliding off of the pen injector;

FIGS. 15A and 15B show a variant of the adapter—pen injector configuration of FIGS. 14A and 14B;

FIG. 16 is a cross-sectional view of an adapter configured for external sealing of its flow path arrangement;

FIG. 17 is a cross-sectional view of an adapter with a wide flow path arrangement;

FIGS. 18 and 19 illustrate an adapter and a matching cap with a recessed edge;

FIGS. 20A-21B illustrate a dosed drug delivery device and a matching cap that interacts with the dosed drug delivery device to facilitate intradermal injection of a liquid;

FIG. 22 illustrates a fluid delivery device that includes a microneedle covered by a cap and a spring that keeps the microneedle from emerging via an aperture in the cap until the device is used for injection;

FIGS. 23A and 23B show an adapter and a corresponding pen injector with a “bayonet”-style interface.

### DESCRIPTION OF THE PREFERRED EMBODIMENTS

The present invention is an adapter for use with a dosed drug delivery device to achieve intradermal dosed delivery of a liquid.

The principles and operation of adapters according to the present invention may be better understood with reference to the drawings and the accompanying description.

By way of introduction, the present invention relates to an adaptation of a microneedle drug delivery interface and corresponding technique described in US Patent Application Publication No US 2005/0209566 A1 to render it suitable for use as a disposable drug delivery interface for pen injectors. The adapter most preferably employs microneedles produced by MEMS techniques from a single-crystal block of material such as silicon according to the teachings of U.S. Pat. No. 6,533,949. Alternatively, various other forms of microneedles and/or other materials may be used, such as are taught in U.S. Pat. No. 6,503,231 to Prausnitz et al. These documents are hereby incorporated by reference herein and provide helpful background to the present invention.

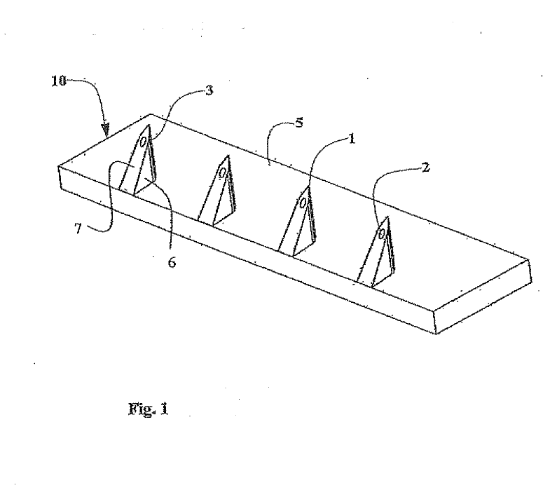

Referring now to the drawings, FIG. 1 shows a particularly preferred implementation of a linear array 10 of microneedles for use in the adapter of the present invention. Specifically, linear array 10 includes a number of hollow microneedles, typically between 1 and 10, more preferably between 3 and 6, and in the preferred case illustrated here, 4. Each microneedles has a penetrating point 1, a liquid flow channel 2 and preferably also a cutting edge 3. The microneedles are preferably integrally formed with a substrate 5, having a substantially planar surface. In the preferred implementation shown here, each microneedle is formed by a set of one or more walls 6 standing substantially upright from the substantially planar surface of substrate 5, and an inclined surface 7 intersecting with walls 6. Flow channel 2 is preferably formed as a bore passing through substrate 5 and intersecting with inclined surface 7. The linear microneedle array 10 is preferably formed using a combination of dry etching and wet etching processes from a single crystal of material, most preferably silicon, by techniques such as those described in detail in the aforementioned U.S. Pat. No 6,533,949. Preferred dimensions for the microneedles for this application are a total height in the range of 250 to 650 microns, and most preferably 450±30 micron. The flow channel 2 may be round or of other cross-sectional shape, and preferably has a minimum internal diameter of about 45±10 microns if round, and an equivalent minimum cross-sectional area if otherwise shaped.

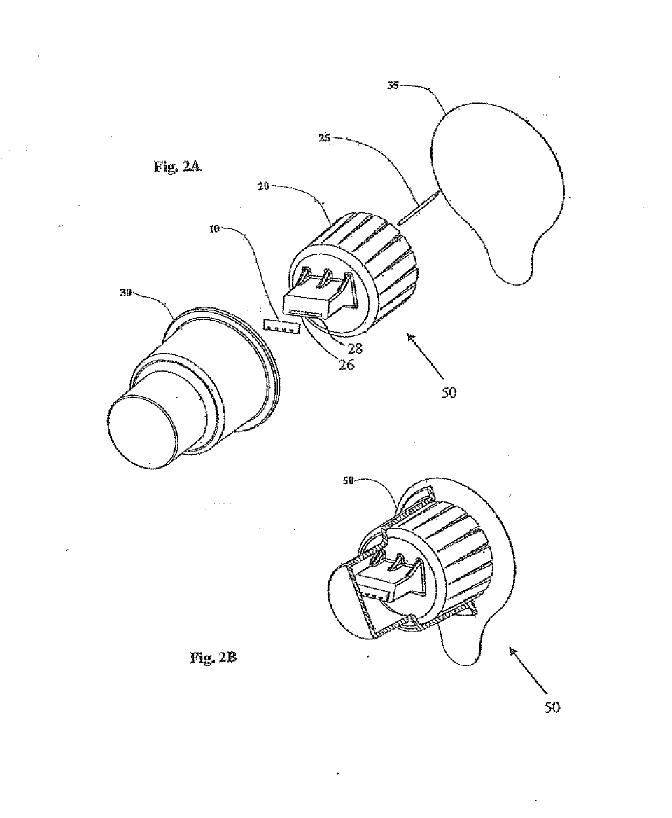

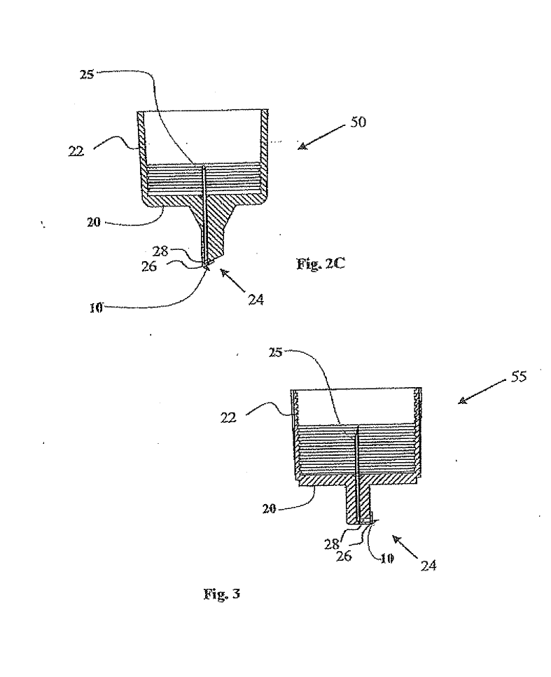

Turning now to FIGS. 2A-2C, these illustrate a first preferred embodiment of an adapter, generally designated 50, constructed and operative according to the teachings of the present invention, for achieving intradermal dosed delivery of a liquid by use of a dosed drug delivery device. Generally speaking, adapter 50 includes a connector including an attachment configuration 22 for attachment to a dosed drug delivery device, and a hollow needle 25 deployed for piercing a septum of a reservoir (typically, a cartridge such as a shell vial with moveable plug) of the dosed drug delivery device. Adapter 50 also features a liquid delivery interface 24, mechanically linked to the connector, including a substantially straight skin contact edge 26 and linear array 10 of hollow microneedles deployed substantially adjacent to, and arrayed substantially parallel to, skin contact edge 26. The microneedles preferably projecting away from the skin contact edge 26. A flow path arrangement 28 interconnects needle 25 with the flow channels of the microneedles in linear array 10.

Prior to use, adapter 50 is preferably protected by a front cover 30, as shown in FIGS. 2A and 2B, which protects the microneedles from accidental mechanical damage. Front cover 30 preferably also seals against a peel-off backing sheet 35 which prevents accidental contact with needle 25 and maintains sterility, together forming a pre-sealed sterile packaging for adapter 50. The device can be sterilized using common methods such as

Gamma irradiation or exposure to Ethylene Oxide.

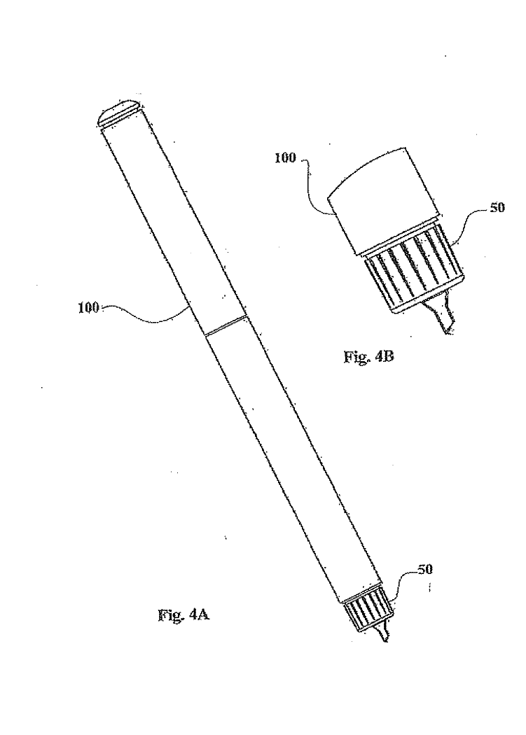

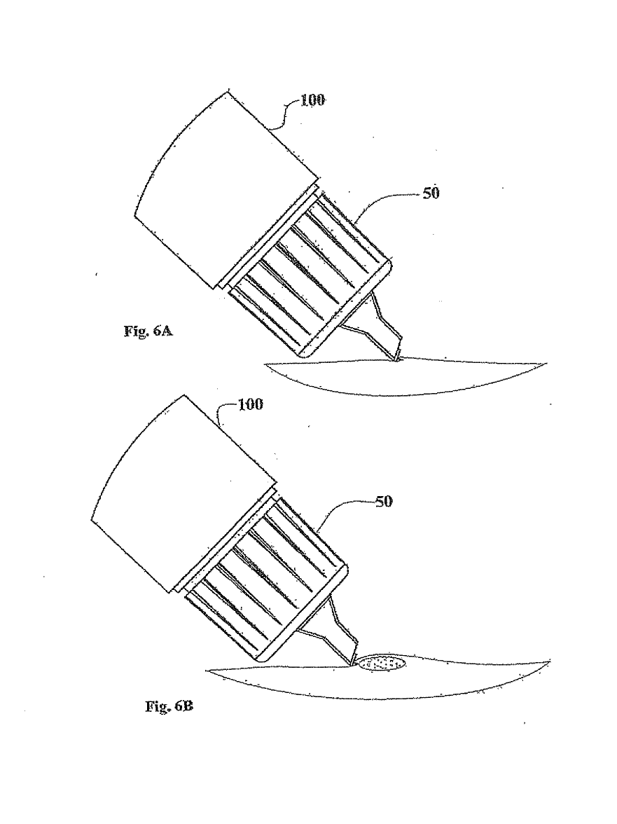

The mode of use of adapter 50 will be understood with reference to FIGS. 4A, 4B, 6A and 6B. First, after removal of backing sheet 35, attachment configuration 22 is attached instead of a pen needle to a conventional pen injector 100, as shown in FIG. 4A and enlarged in FIG. 4B. As adapter 50 is attached to the pen injector, needle 25 (not visible in this view) pierces the septum of the liquid vial or cartridge within the pen injector, thereby forming a flow connection to the microneedles. Then, as shown in FIG. 6A, the assembled device is brought into contact with the user's skin and pushed gently with a vector of motion having a non-zero component parallel to the initial surface of the skin (to the right as shown) so as to achieve penetration with the microneedles projecting primarily sideways, parallel to the initial surface of the skin. This form of insertion achieves numerous advantages over conventional perpendicular insertion, as detailed in the aforementioned. US Patent Application Publication No. US 2005/0209566 A1. The pen injector is then actuated in the normal manner to achieve injection of the desired dose of the contained liquid, as illustrated schematically in FIG. 6B.

At this stage, it will already be apparent that the adapter of the present invention provides profound advantages over the prior art. Specifically, all pen injector art known to the inventors maintains the conventional approach of perpendicular insertion of the needle(s) into the skin, thereby suffering from the aforementioned limitations of penetration depths in excess of 1 millimeter for conventional needles, or problems of incomplete penetration and ejection by back pressure for microneedles. In contrast, by providing the unique geometry of the present invention in which an array of microneedles are adjacent to a skin contact edge, the present invention facilitates insertion of microneedles so that the microneedle flow channels are directed sideways, i.e., at an angle in the range of ±30° from the initial plane of the skin surface, into tissue not squashed under the device. As a result, the adapter of the present invention allows a pen injector to be used to achieve shallower intradermal liquid delivery than is possible using conventional devices, and is believed to encounter reduced flow impedance and achieve better intradermal distribution than would otherwise be achieved. These and other advantages of the present invention will be better understood with reference to the following description.

Turning now to the features of the present invention in more detail, skin contact edge 26 is preferably formed as an edge of a block of material which supports the microneedle array 10. Most preferably, this block is integrally formed with at least part of the attachment configuration. Thus, in the example of FIGS. 2A-2C, adapter 50 is most preferably formed from a combination of only three elements: microneedle array 10, needle 25, and a unitary body 20 formed from molded polymer material which provides both the attachment configuration (in this case, a threaded collar) and support for microneedle array 10. Most preferably, body 20 is formed from molded polycarbonate. This three-element implementation minimizes production costs, rendering the adapter suitable for disposable use as a pen needle substitute.

Body 20 also preferably defines any flow paths 28 required to interconnect needle 25 with the flow channels of the microneedles. In the preferred implementation shown, this includes a transverse open channel formed under the point of attachment of microneedles array 10 so that, when the substrate is attached by use of adhesive, welding or other known methods, the channel together with the rear surface of the substrate forms a closed channel for distributing liquid from needle 25 to all of the microneedles. The positioning of this channel is chosen to intersect a central axis of the adapter 50 along which needle 25 is aligned, thereby simplifying manufacture of body 20, as will be clear to one familiar with plastic injection molding technology.

The form of body 20 is chosen to facilitate bringing the microneedles into contact with the skin in the correct orientation. In the preferred example shown here, body 20 is formed with a forward projecting portion which is roughly rectangular in cross-section, having a major dimension parallel to the extensional direction of microneedle array 10 and a minor dimension perpendicular thereto. The microneedles are preferably deployed with the inclined surface having flow channel 2 facing downwards, i.e., inwards towards the depth of the tissue.

In order to optimize the sideways insertion geometry, the microneedles are preferably close to edge 26. Preferably, a distance between skin contact edge 26 and each of the microneedles, defined as the distance between edge 26 and the closest part of the base of the microneedles, is no greater than the height of the microneedles themselves as measured perpendicular to the surface of the substrate. Most preferably, the microneedles are juxtaposed with their base starting substantially at edge 26. Parenthetically, it should be noted that edge 26 itself may be provided by either the edge of the substrate of microneedle array 10 or by an edge of body 20 adjacent to the array 10.

It will be noted that adapter 50 causes a significant deflection of the flow direction between the axial direction of the dosed drug delivery device (corresponding to the direction of needle 25) and the injection direction as defined by the flow channels of the microneedles.

This deflection is preferably at least about 20 degrees and, more preferably, between about 30 and about 150 degrees. In the case shown here, the deflection is roughly 40 degrees. Nevertheless, in order to achieve an injection direction near parallel to the initial plane of the skin, this embodiment requires deployment of the pen injector at an inclination as shown in FIGS. 4A, 4B, 6A and 6B.

Parenthetically, although the device is illustrated here in a preferred embodiment in which a linear array of microneedles is used, it should be noted that a minimal embodiment in which a single microneedle is used in proximity to skin contact edge 26 also falls within the broad scope of the present invention.

FIGS. 3, 5A, 5B, 7A and 7B illustrate an alternative embodiment of an adapter, generally designated 55, which provides a larger deflection of the flow direction, namely, about 90 degrees. This orientation achieves sideways injection while allowing the device to be held generally orthogonally to the initial skin surface, in a manner more similar to the orientation of a pen injector used with a conventional pen needle. Initial insertion of the microneedles into the skin surface is preferably performed at a slight angle, as illustrated in

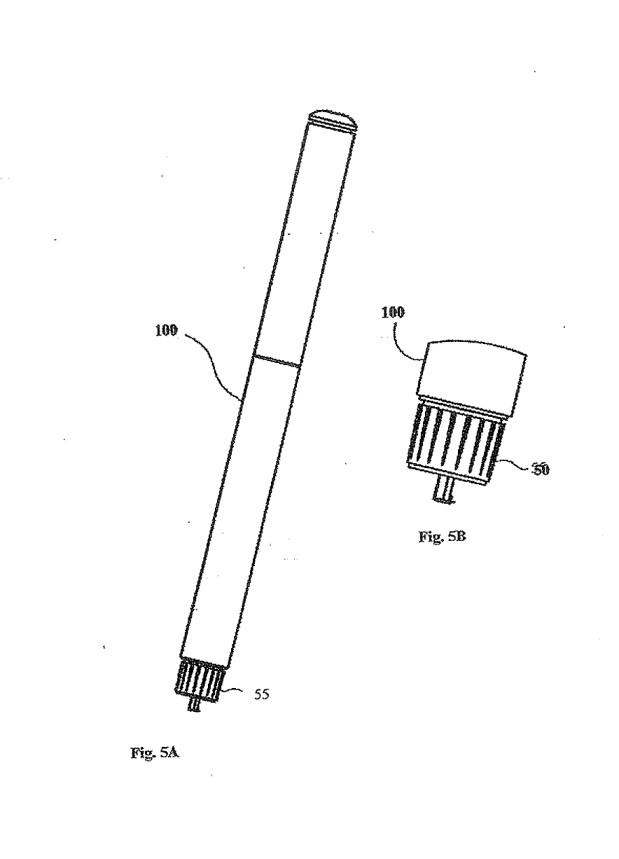

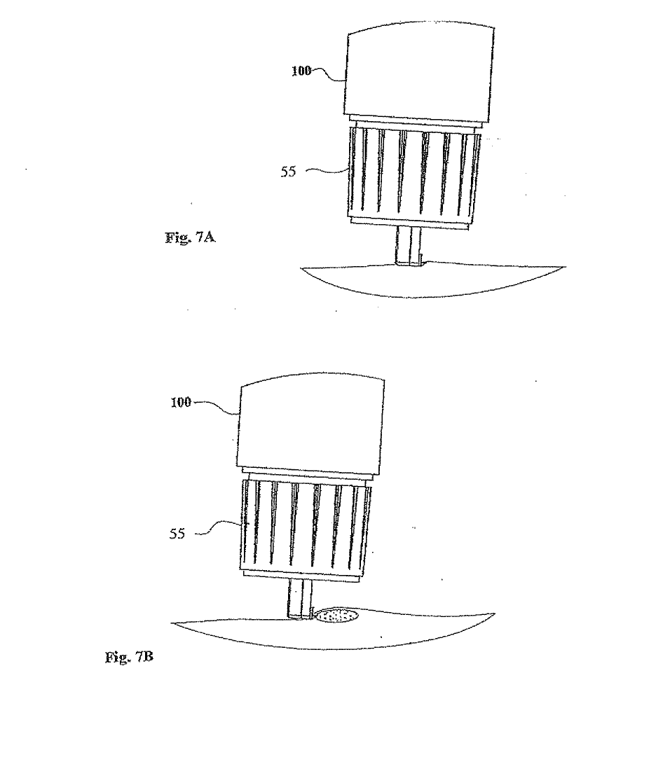

FIGS. 5A and 5B, and typically requires a slight turning motion, applying an anticlockwise turning moment in the orientation as illustrated in FIG. 7A.

In other respects, the structural features and function of adapter 55 will be understood by analogy to the corresponding features and function of adapter 50 described above, with like elements being labeled similarly.

The embodiments described above did not specify a rotational orientation of adapter 50 or 55 relative to pen injector 100. It often is advantageous to specify such an orientation. For example, as pen injector 100 is being used with adapter 50 as illustrated in FIGS. 6A and 6B for intracutaneous injection of a liquid, if pen adapter 100 includes a scale for dose measurement or a dialer to determine the dose of liquid being delivered, it may be advantageous for the user of pen injector 100 to be able to see these features of pen injector 100 as the user is injecting the liquid. In that case, the adapter should be such that these features of pen injector 100 must be on the upper surface of pen injector 100, and so visible to the user, when microneedle array 10 is oriented relative to pen injector 100 as illustrated in FIGS. 6A and 6B.

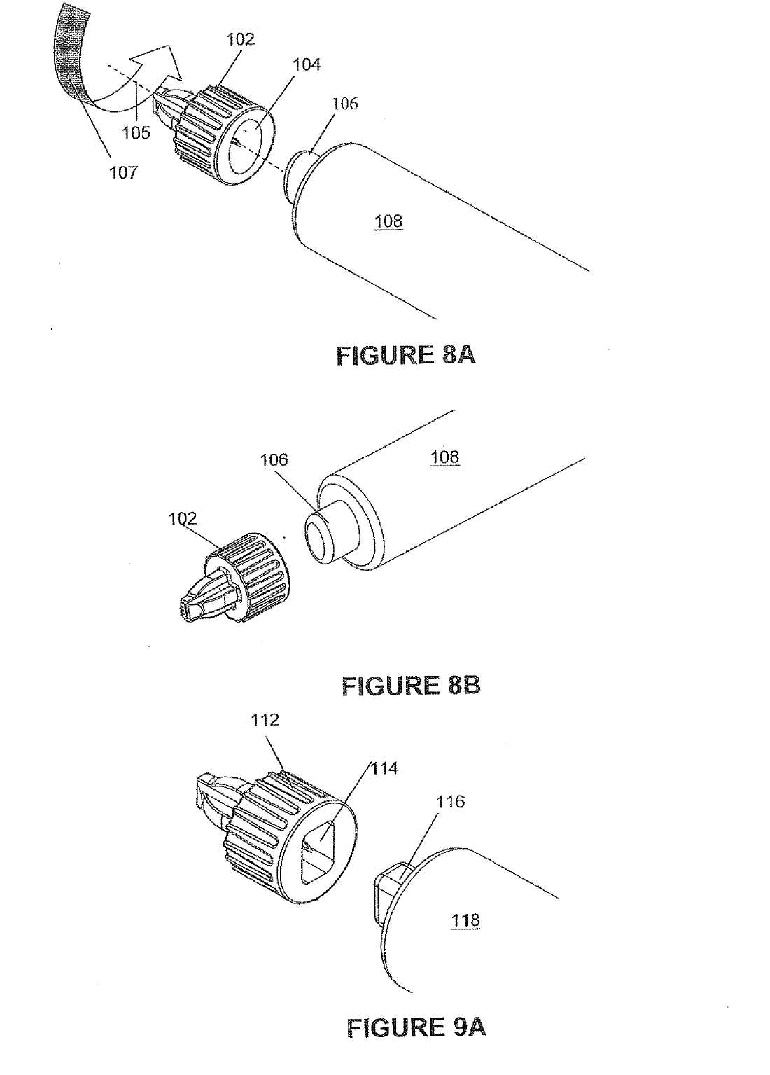

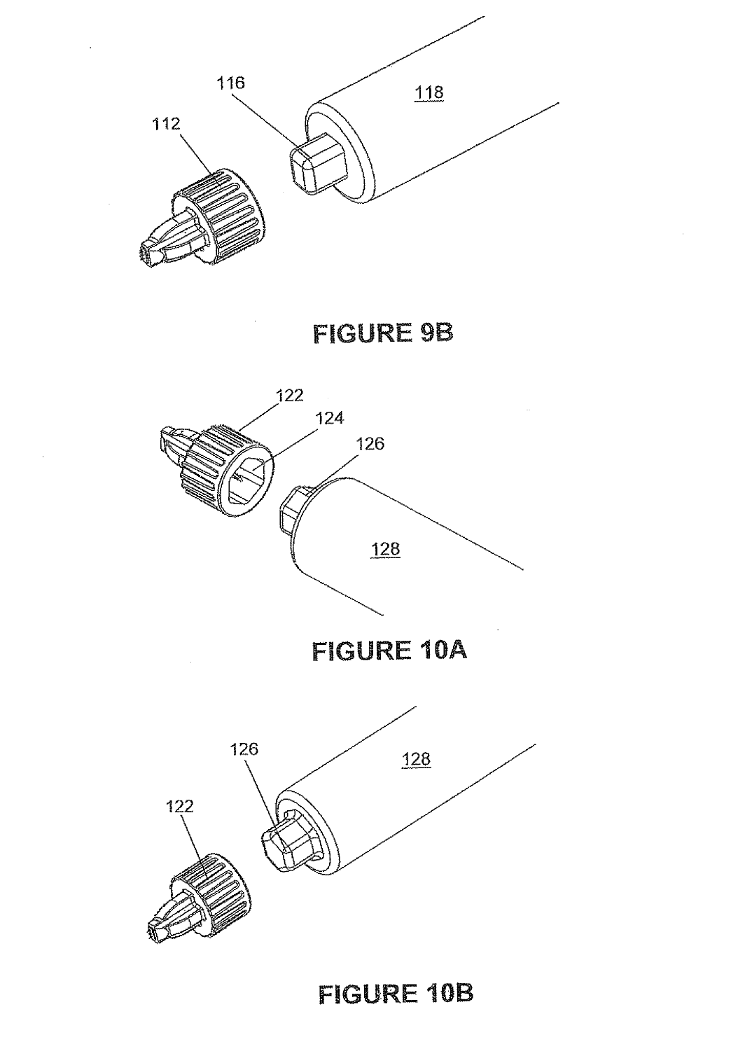

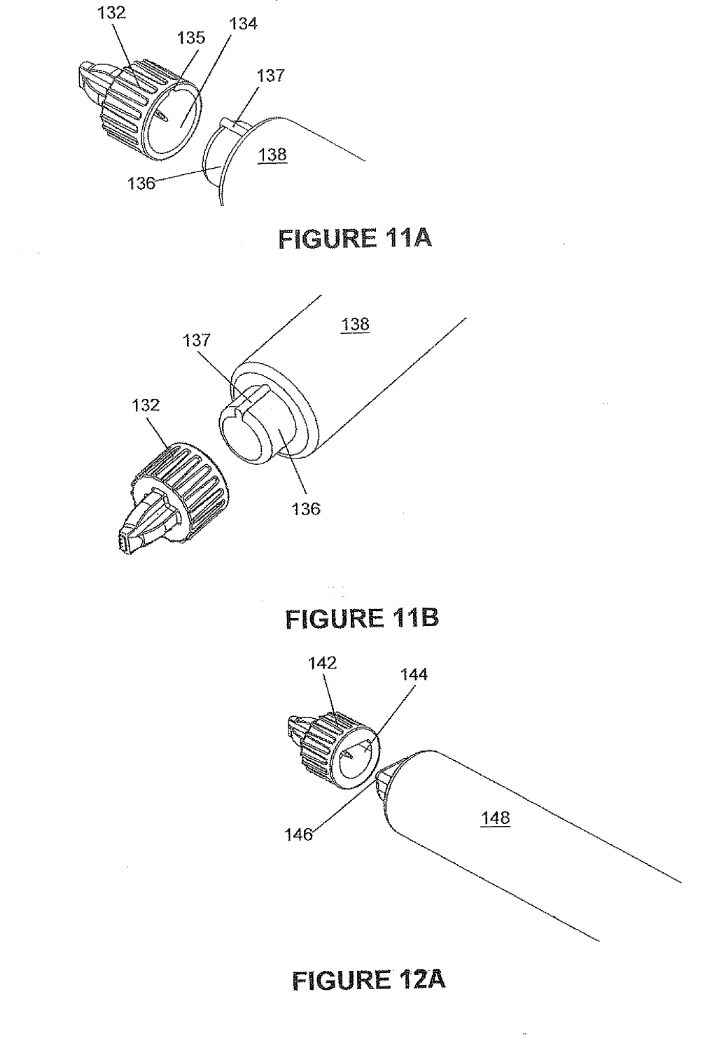

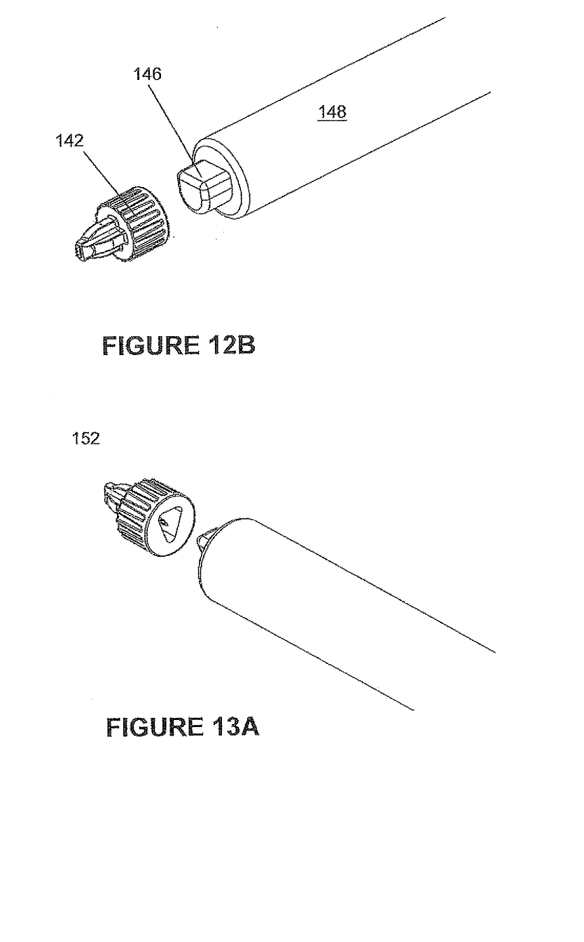

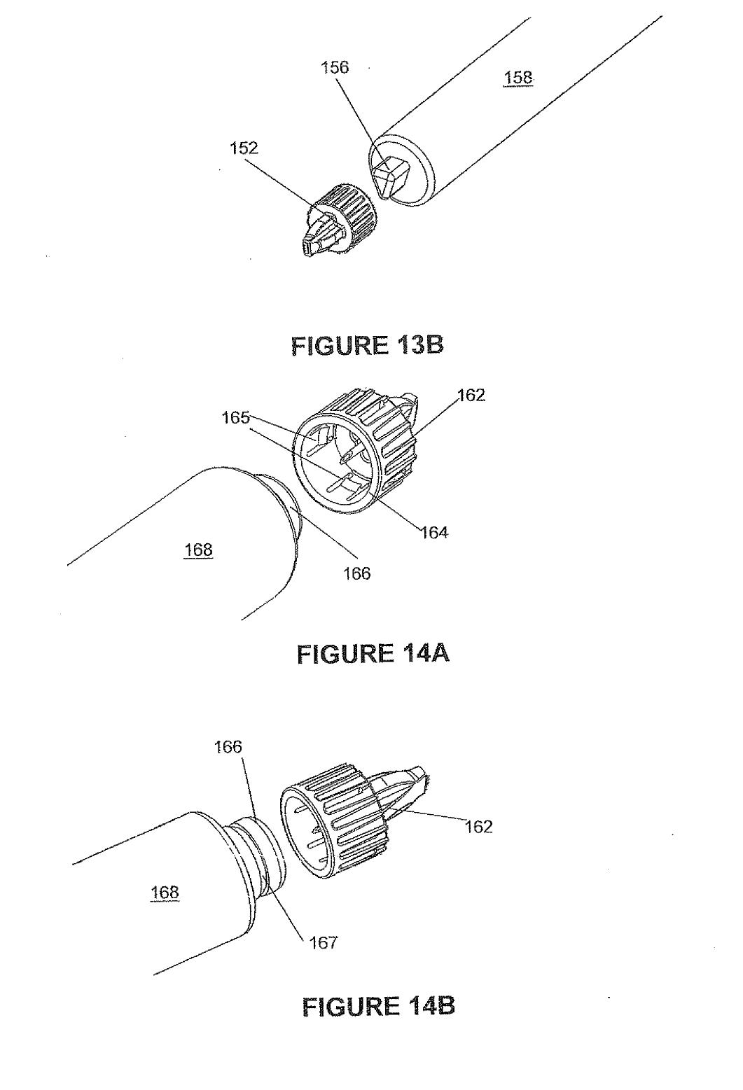

FIGS. 8A-13B illustrate adapters and pen injectors that are configured to restrict the rotational orientation of the adapters relative to the pen injectors. FIGS. 8A and 8B show an adapter 102 with an elliptical recess 104 that mates with a matching elliptical projection 106 at the distal end of a pen injector 108. FIGS. 9A and 9B show an adapter 112 with a rectangular recess 114 that mates with a matching rectangular projection 116 at the distal end of a pen injector 118. FIGS. 10A and 10B show an adapter 122 with a hexagonal recess 124 that mates with a matching hexagonal projection 126 at the distal end of a pen injector 128. Hexagonal recess 124 and hexagonal projection 126 are examples of regular polygonal recesses and projections. FIGS. 11A and 11B show an adapter 132 whose otherwise cylindrical recess 134 includes a notch 135 that accommodates a tab 137 of an otherwise cylindrical projection 136 of a pen injector 138. FIGS. 12A and 12B show an adapter 142 with a truncated cylindrical recess 144 that mates with a matching truncated cylindrical projection 146 at the distal end of a pen injector 148. FIGS. 13A and 13B show an adapter 152 with an isosceles triangular recess 154 that mates with a matching isosceles triangular projection 156 at the distal end of a pen injector 158. Adapters 102 and 112 are restricted to only two rotational orientations relative to their pen injectors 108 and 118. Adapter 122 is restricted to only six rotational orientations relative to its pen injector 128. In general, the number of rotational orientations to which an adapter with a regular polygonal recess is restricted, relative to its pen injector, is equal to the number of sides of the polygon. Adapters 132, 142 and 152 are restricted to only one rotational orientation relative to their respective pen injectors 138, 148 and 158.

In FIG. 8A, dashed line 105 indicates the axis of approximate rotational symmetry of adapter 102 and pen injector 108. The two rotational orientations of adapter 102 are relative to axis 105, as indicated by arrow 107. The rotational orientations of adapters 112, 122, 132, 142 and 152 are defined similarly.

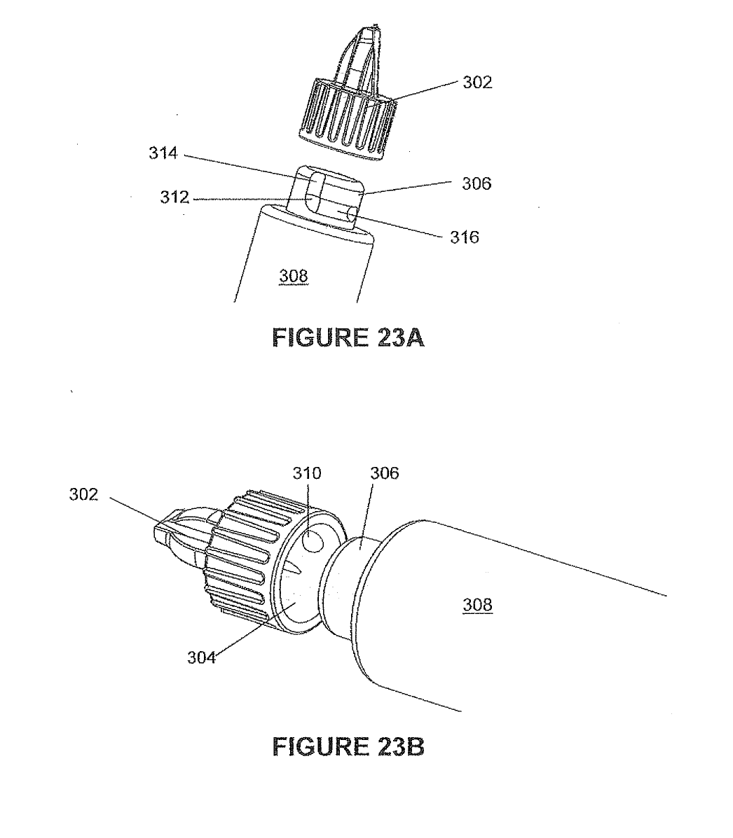

Adapter 102 is mounted on pen injector 108 by sliding adaptor 102 axially, i.e. in the direction defined by axis 105, towards pen injector 108 so that recess 104 mates with projection 106, and similarly for how adapters 112, 122, 132, 142 and 152 are mounted on pen. injectors 118, 128, 138, 148 and 158. Other mechanical arrangements for sliding an adapter onto a pen injector so as to be mounted only in one of a small number of preferred orientations also are possible. One such arrangement is illustrated in FIGS. 23A and 23B that show an adapter 302 with a cylindrical recess 304 and a pen adapter 308 with a cylindrical projection 306. A bump 310 that protrudes into recess 304 prevents adapter 302 from being slid onto projection 306 unless bump 310 is aligned with the vertical leg 314 of an L-shaped track 312 in projection 306. When bump 306 reaches the end of vertical leg 314, adapter 302 is turned to slide bump 306 along the horizontal leg 316 of track 312 to place adapter 302 in its proper (single) orientation relative to pen adapter 308.

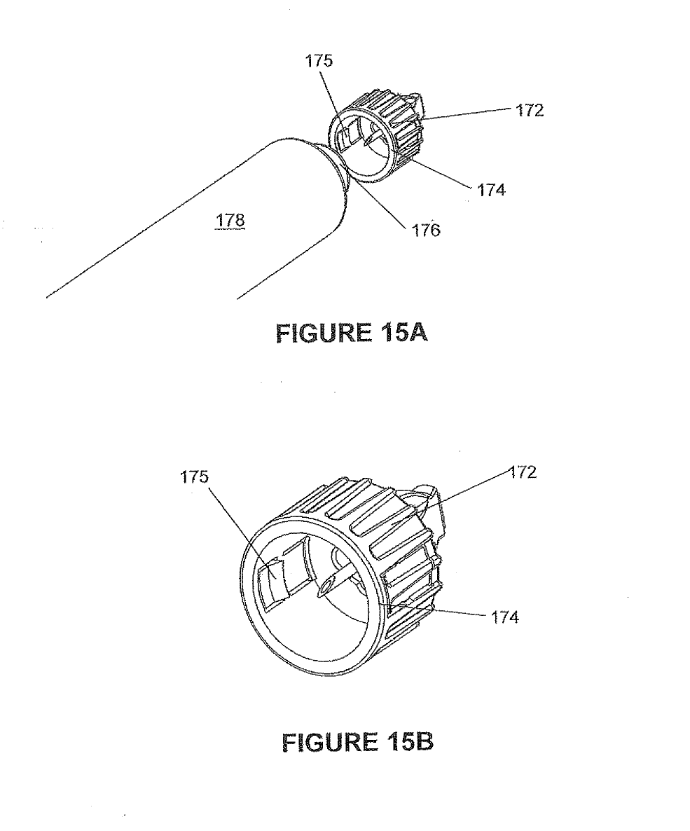

Unlike adapters 50 and 55, adapters 102, 112, 122, 132, 142 and 152 can slide off of their respective pen injectors 108, 118, 128, 138, 148 and 158 as easily as they can slide on. FIGS. 14A and 14B show an adapter 162 with a cylindrical recess 164 and a pen injector 168 with a cylindrical projection 166 that are configured to prevent this. Adapter 162 has tabs 165 that, when adapter 162 is slid onto projection 166, latch into a shelf 167 that surrounds projection 166. FIGS. 15A and 15B show a similar adapter 172—pen injector 178 combination in which adapter 172 includes only one tab 175 that latches onto a shelf (not shown) that surrounds projection 176 of pen adapter 178 that mates with recess 174 of adapter 172. Adapters 162 and 172 are free to swivel rotationally about projections 166 and 176 rather than being fixed in a specific rotational orientation with respect to pen injector 168 and 178; but similar mechanisms in adapters 102, 112, 122, 132, 142 and 152 and their respective pen injectors 108, 118, 128, 138, 148 and 158 prevent adapters 102, 112, 122, 132, 142 and 152 from slipping off their respective pen injectors 108, 118, 128, 138, 148 and 158.

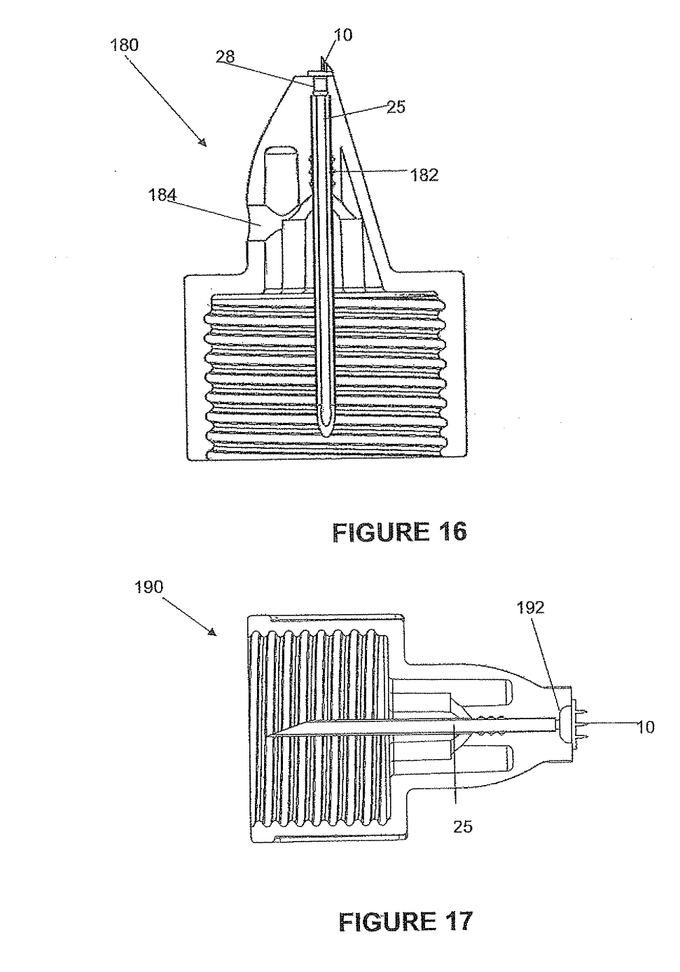

As described above, the septum-piercing needle and the skin-penetrating needle of a prior art pen needle typically are implemented as opposite ends of a single double-ended needle. As such, there is no danger of liquid leaking from a gap between the septum-piercing needle and the skin-penetrating needle. This is not the case for the adapters of the present invention, in which hollow needle 25 and microneedle array 10 are separate components that are connected via flow path arrangement 28. It is especially important to prevent leakage around flow path arrangement 28 in the typical case that the dose of liquid to be delivered via microneedle array 10 is very small. For this purpose, a sealant is introduced to the adapter to seal the interfaces between microneedle array 10, flow path arrangement 28 and needle 25. The sealant is introduced as a liquid that hardens in place to seal the interfaces between microneedle array 10, flow path arrangement 28 and needle 25. FIG. 16 is an axial cross-section of an adapter 180 that includes two features for promoting the adhesion of a sealant to flow path arrangement 28 and needle 25 inside adapter 180. The first feature is a roughening 182 of the outer surface of needle 25 to promote adhesion of the sealant to needle 25. Methods of roughening the outer surface of needle 25 are known in the art, and include laser treatment, ultrasonic treatment and powder blasts. The second feature is a port 184 in the side of adapter 180 to facilitate introduction of the sealant as a liquid into the interior of adapter 180. [I didn't claim or illustrate anything about “a long channel in hob to ensure wide bonding area” because in order to claim that we need to. quantify what we mean by “long”, either in relative terms (e.g. relative to the diameter of the hub) or in absolute terms.]

Even a well-sealed flow path arrangement 28 could be breached by high pressure and turbulence in the liquid as the liquid is injected. FIG. 17 shows an adapter 190 whose flow path arrangement 192 has been made wider, transversely to the direction of flow of the liquid from needle 25 to microneedle array 10, than both needle 25 and the portion of substrate 5 of microneedle array 10 that is occupied by the microneedles themselves. It is believed that this widening of flow path arrangement 192 mitigates the pressure and turbulence of the liquid as the liquid is injected via microneedle array 10, thereby inhibiting or preventing leakage from the interface between needle 25 and flow path arrangement 192 and from the interface between flow path arrangement 192 and microneedle array 10. FIG. 17 shows flow path arrangement 192 widened in the transverse direction parallel to microneedle array 10. Additionally or alternatively, flow path arrangement 192 could be widened in the transverse direction perpendicular to microneedle array 10.

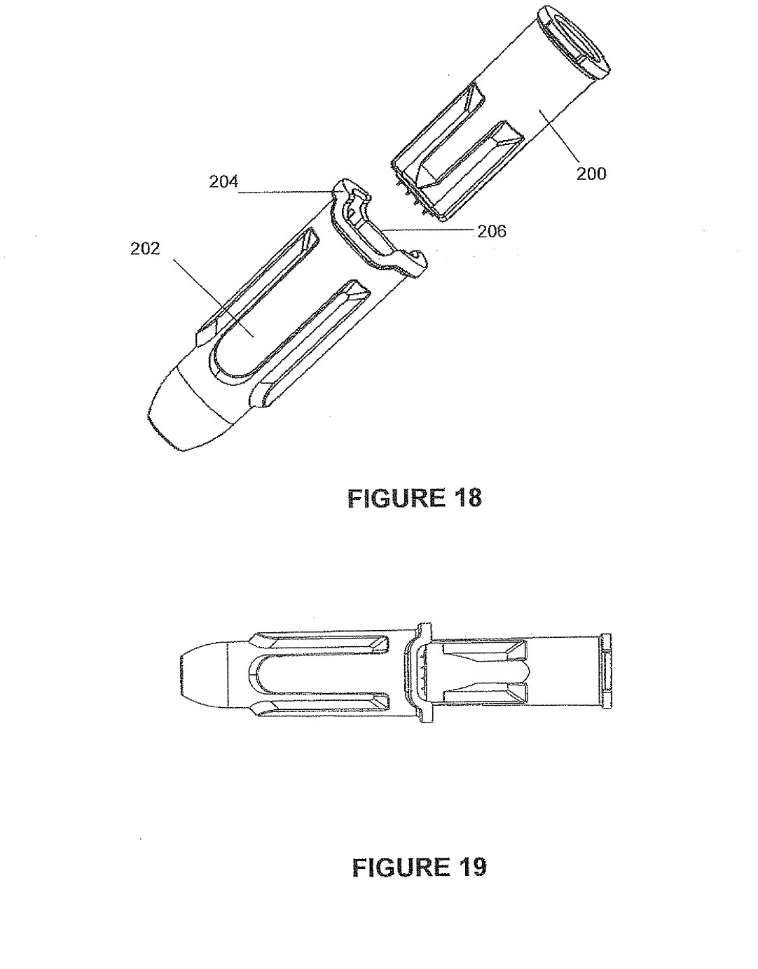

FIGS. 18-21B illustrate innovative caps for adapters and for pen injectors to which adapters have been attached.

Returning temporarily to FIGS. 2A and 2B to explain the need for the cap-adapter arrangement illustrated in FIG. 18, adapter 50 normally is attached to pen injector 100 by removing backing sheet 35 with cap 30 still in place and screwing adapter 50 onto pen injector 100. When the user is ready to use pen injector 100, the user pulls cap 30 off of adapter 50. If the user pulls cap 30 straight off of adapter 50 (arrow 60), the edge of cap 30 does not contact the microneedles and the microneedles are not damaged. But if while pulling cap 30 off of adapter 50, the user turns cap 30 upward (arrow 65), the edge of cap 30 may hit and damage the microneedles.

FIG. 18 shows an adapter 200 whose matching cap 202 is shaped to prevent such damage. The edge 204 of cap 202 is recessed (206) where edge 204 passes the microneedles as cap 202 is removed from adapter 200. To make sure that cap 202 is placed on adapter 200 in the correct orientation, adapter 200 is made longer in the transverse direction parallel to the skin contact edge of the liquid delivery interface of adapter 200 than in the transverse direction perpendicular to the skin contact edge of the liquid delivery interface of adapter 200. FIG. 19 shows that the short, unrecessed portion of edge 204 continues to grip adapter 200 as recess 206 is withdrawn past the microneedles, so that even if cap 202 is turned into or out of the plane of FIG. 19 at the final stage of withdrawing cap 202 from adapter 200, edge 204 does not contact the microneedles.

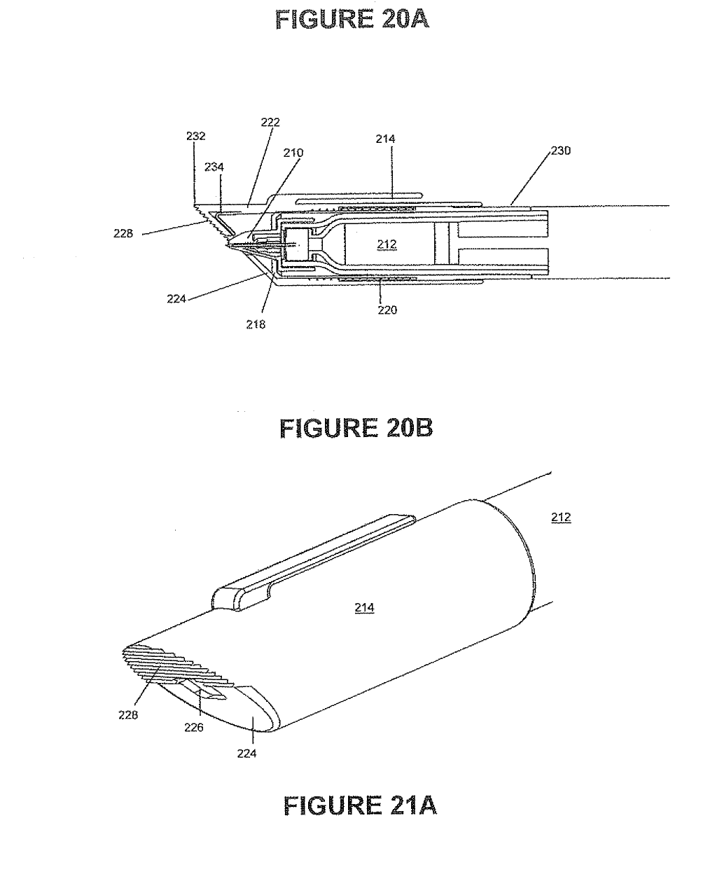

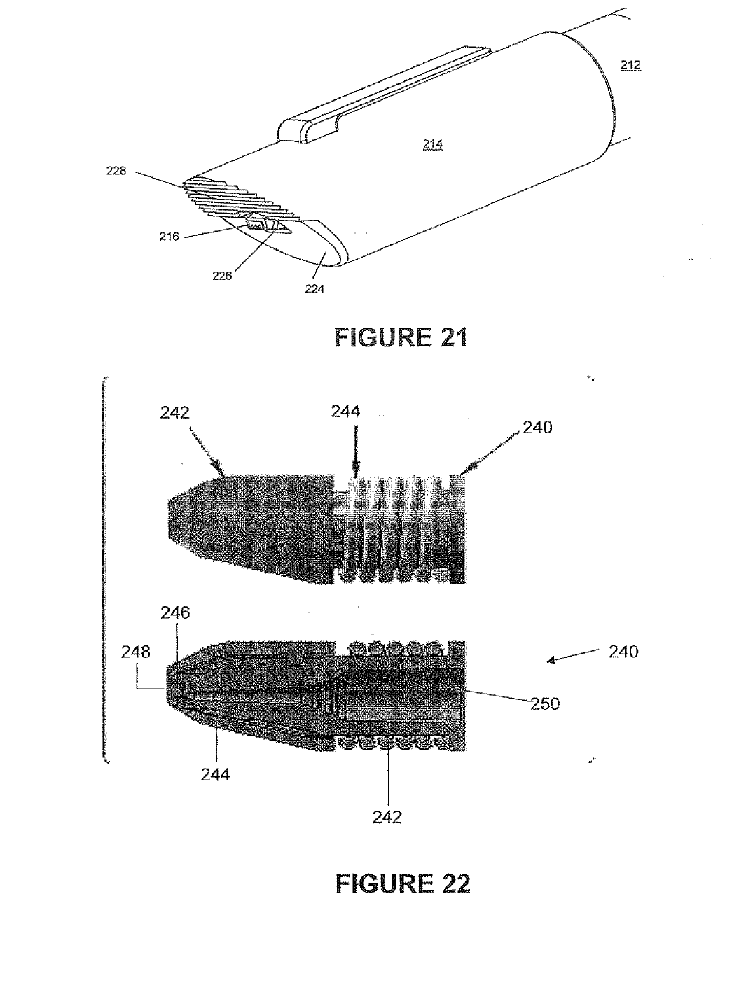

FIGS. 20A and 20B are axial cross-sections of an adapter (210)—pen injector (212) combination equipped with a cap 214 for covering the end of the combination that bears adapter 210 and its microneedles 216. The interior of the body 222 of cap 214 is shaped to conform to pen injector 212 when the end of pen injector 212 that bears adapter 210 is inserted into the proximal end 230 of cap 214. Cap 214 is capped at its distal end 232 by a tilted planar cap cover 224 that includes an aperture 226. One side of the outer surface of cap cover 224 is roughened (228) by being formed with parallel furrows. When the end of pen injector 212 that bears adapter 210 is inserted into the proximal end 230 of cap 214, adapter 210 encounters a spring (220)—loaded retainer 218 that prevents further free movement of adapter 210 towards cap cover 224, as show in FIG. 20A. When retainer 218 is in the position shown in FIG. 20A, a flexible barrier 234 inside cap 214 covers aperture 226 and keeps dirt. from entering cap 214. FIG. 20B shows that further pushing of pen injector 212 against the resistance of spring 220 pushes barrier 234 out of the way of microneedles 216 and pushes microneedles 216 through aperture 226. FIGS. 21A and 21B are perspective exterior views of cap 214 on pen injector 212, with microneedles 216 either concealed inside cap 214 (FIG. 21A) or emerging from aperture 216 (FIG. 21B). The device illustrated in FIGS. 20A-21B is used for injecting a liquid from pen injector 212 into the skin of a user by placing cap cover 224 on the skin and pushing pen injector 212 so that microneedles 216 emerge from aperture 226 and penetrate the skin. Cap cover 224 is tilted so that roughened portion 228 contacts the skin before microneedles 216 contact the skin. Roughened portion 228 of cap cover 224 applies tension to the skin to facilitate the penetration of the skin by microneedles 216.

The principles of cap 214 may be applied more widely than to just pen injectors. FIG. 22 shows, in both external view and cross-sectional view, a microneedle-equipped fluid delivery device 240, similar to those taught in U.S. Pat. No. 7,998,119, configured with a female Luer-Slip fitting 250 for attachment to a syringe. Device 240 includes a spring (242)—loaded cap 244 that conceals microneedles 246 until the syringe to which device 240 is attached is ready to be used. To use the syringe to deliver an injection to a target, the user pushes the syringe against spring 242, causing microneedles 246 to emerge from an aperture 248 in the front of cap 244.

It will be appreciated that the present invention may be used to advantage in a large number of drug delivery applications, including both applications for which pen injectors are conventionally used and new applications for which the shallow intradermal delivery achieved by the present invention may be advantageous. Examples of applications include, but are not limited to, administering: insulin, fertility hormones, growth hormone and vaccines. Other applications include, but are not limited to, the substances and modes of treatment mentioned in US Patent Application Publication No. 2005/0163711 A1, which is hereby incorporated by reference herein.

It will be appreciated that the above descriptions are intended only to serve as examples, and that many other embodiments are possible within the scope of the present invention as defined in the appended claims.

What is claimed is:

1. An adapter for achieving intradermal dosed delivery of a liquid by use of a dosed drug delivery device, the dosed drug delivery device including a reservoir having a pierceable septum, the adapter comprising:
(a) a connector including:
(i) an attachment configuration for attachment to the dosed drug delivery device by sliding the adapter onto the dosed drug delivery device so that a recess of the adapter mates with a matching projection of the dosed drug delivery device, and
(ii) a hollow needle deployed for piercing the septum;
(b) at least one hollow microneedle projecting from said connector; and
(c) a flow path arrangement interconnecting said needle and said at least one hollow microneedle;
wherein said recess is shaped to restrict said at least one microneedle to one of a finite number of rotational orientations relative to an axis of the dosed drug delivery device when the adapter is attached to the dosed drug delivery device.

2. The adapter of claim 1, wherein said at least one microneedle is included in a liquid delivery interface that is mechanically linked to said connector, said liquid delivery interface including a substantially straight skin contact edge, said at least one microneedle being deployed substantially adjacent to said skin contact edge and projecting away from said skin contact edge.

3. The adapter of claim 1, wherein said at least one microneedle is restricted to a single said orientation.

4. The adapter of claim 1, comprising a linear array of said microneedles.

5. A combination of the adapter of claim I with a dosed drug delivery device, the combination further including a dosed drug delivery device having a liquid reservoir including a pierceable septum, the dosed drug delivery device having a projection that matches said recess, the adapter having been connected to said dosed drug delivery device by the adapter being slided onto the dosed drug delivery device so that said recess mates with said projection and so that said hollow needle pierces said septum thereby bringing said at least one microneedle into flow connection with contents of said reservoir.

6. The adapter of claim 1, wherein said attachment configuration includes a mechanical arrangement for inhibiting removal of the adapter from the dosed drug delivery device after the adapter has been attached to the dosed drug delivery device.

7. (canceled)

8. The adapter of claim 74, wherein said at least one microneedle is included in a liquid delivery interface that is mechanically linked to said connector, said liquid delivery interface including a substantially straight skin contact edge, said at least one microneedle being deployed substantially adjacent to said skin contact edge and projecting away from said skin contact edge.

9-19. (canceled)
